(19) United States
(12) Patent Application Publication
(10) Pub. No.: US 2005/0171449 A1
(43) Pub. Date: Aug. 4, 2005

(54) Method and apparatus for detecting ammonia from exhaled breath

(76) Inventors: Kenneth S. Suslick, Champaign, IL (US); Keren I. Hulkower, Evanston, IL (US); Avijit Sen, Champaign, IL (US); Mitchell A. Sroka, Philo, IL (US); William B. McNamara III, Ogden, IL (US)

Correspondence Address: Jane Massey Licata;Licata & Tyrrell P.C., 66 E. Main Street, Marlton, NJ, 08053, US

(21) Appl. No.: 11/058,497

(22) Filed: Feb. 15, 2005

Related U.S. Application Data
(63) Continuation-in-part of application No. 10/279,788, filed on Oct. 24, 2002.
Application No. 10/279,788 is a continuation-in-part of application No. 09/705,329, filed on Nov. 3, 2000, now Pat. No. 6,495,102.
Application No. 09/705,329 is a continuation-in-part of application No. 09/532,125, filed on Mar. 21, 2000, now Pat. No. 6,368,558.

(51) Int. Cl. A61B 5/08; G01N 21/00; G01N 21/75

(52) U.S. Cl. 600/532; 422/55; 436/164

(57) ABSTRACT

The present invention is an apparatus for detecting the presence of exhaled ammonia and a method for using the same to detect the present of a Helicobacter in a subject. The apparatus is composed of a breath capture device having a sensor plate with at least one selected Lewis acid dye (e.g., a metal ion-containing dye such as a metalloporphyrin) deposited thereon which produces a detectable spectral, transmission or reflectance response in the presence of ammonia.

### INTRODUCTION

This application is a continuation-in-part application of U.S. patent application Ser. No. 10/279,788, filed Oct. 24, 2002, which is a continuation-in-part application of U.S. patent application Ser. No. 09/705,329, filed Nov. 3, 2000, now U.S. Pat. No. 6,495,102, which is a continuation-in-part application of U.S. patent application Ser. No. 09/532,125, filed Mar. 21, 2000, now U.S. Pat. No. 6,368,558, all of which are incorporated herein by reference in their entireties.

This invention was made in the course of research sponsored by the National Institutes of Health (Grant No. R01-HL25934). The U.S. government may have certain rights in this invention.

### BACKGROUND OF THE INVENTION

Helicobacter pylori is a bacterium that causes chronic inflammation (gastritis) of the inner lining of the stomach in humans. This bacterium also is the most common cause of ulcers worldwide. H. pylori infection is most likely acquired by ingesting contaminated food and water and through person-to-person contact. Infected individuals usually carry the infection indefinitely unless they are treated with medications to eradicate the bacterium. One out of every six patients with H. pylori infection will develop ulcers of the duodenum or stomach. H. pylori also is associated with stomach cancer and a rare type of lymphocytic tumor of the stomach called MALT lymphoma.

Persons infected with H. pylori develop serum antibodies to the organism. These antibodies can be detected in serum by binding the antibodies to purified H. pylori antigen, followed by detection of the human immunoglobulins. Both IgG and IgA immunoglobulin classes are found, and can be used diagnostically. However, the IgG subclass decreases after eradication, and high titers are usually associated with acute infections. Serological testing for H. pylori is only one of a number of diagnostic techniques that can be used.

H. pylori produces an abundant amount of urease. By producing ammonia from urea, H. pylori can elevate the pH of the surrounding environment thereby allowing it to survive in the acidic environment of the stomach. The unique ability of H. pylori to produce an abundant amount of urease has been utilized to identify the presence of the organism in solid tissue specimens, retrieved by invasive biopsy, placed into small test volumes containing urease substrate (CLOtest; U.S. Pat. No. 4,748,113). A gel acts as a support for the tissue specimen and contains the urease substrate and a pH indicator. This test requires up to 24 hours for completion. Methods of using urea and a pH indicator in combination with endoscopic procedures have also been suggested for detecting H. pylori (Thillainayagam, et al. (1991) Gut 32(5): 467-469; Iseki, et al. (1998) Gut 42: 20-23; U.S. Pat. No. 5,556,760; U.S. Pat. No. 6,228,605; U.S. Pat. No. 6,479,278).

A test-strip form of the urease test which provides visual results on solid tissue specimens within an hour or more at room temperature has also been developed (Yousfi, et al. (1997) Am. J. Gastroenterol. 92(6): 997-999; Elitsur, et al. (1998) Am. J. Gastroenterol. 93(2): 217-219; U.S. Pat. Nos. 5,314,804 and 5,420,016). This test-strip also relies, however, on invasive biopsy to obtain the tissue sample.

Non-invasive breath testing methods for detecting H. pylori are also available. Breath test analyzers are known which detect the presence of isotopes of carbon in exhaled carbon dioxide produced by the bacterial urease after urea ingestion, but these tests have a high capital cost inherent in the isotopic analysis. U.S. Pat. No. 4,830,010; U.S. Pat. No. 4,851,209; U.S. Pat. No. 5,848,975; U.S. Pat. No. 6,171,811; U.S. Pat. No. 6,491,643; WO 97/14029; Peura, et al. (1996) Am. J. Gastro. 91: 233-38.

U.S. Pat. No. 4,947,861 teaches a breath test wherein a patient ingests a quantity of urea and after about 10 minutes exhales into breath collection apparatus to provide a breath sample of alveolar air collected during the last part of the exhalation. Immediately upon issuing from the patient, the expired breath is passed in contact with a solid-state body of alkaline hygroscopic material, e.g., sodium hydroxide, that substantially removes water vapor from the breath but is inert to ammonia in the presence of water. The breath sample, thus dehydrated, is delivered to a sensor (e.g., an electrode device or an alkali-indicating material) which signals the presence of ammonia in it, to indicate the presence of H. pylori in the stomach.

U.S. Pat. No. 6,509,169 discloses a method for detecting H. pylori in a subject's gastroenteral tract by measuring a change in resistance of an electronic or electrochemical ammonia sensor, notably a polypyrrole film, on exposure to gas from the subject's lungs and/or stomach.

U.S. patent application Ser. No. 10/294,352 discloses a breath-test method consisting of measuring a basal ammonia level with a highly sensitive calorimetric ammonia sensor, administering non-isotopic urea and continuing measurement of the ammonia content in a plurality of consecutive breaths. This application teaches that the ammonia sensor can be an ammonia sensitive indicator dye such as bromocresol green or bromophenol purple which undergo changes in absorption spectra upon acid/base dissociation. By only measuring the pH response of a single dye, however, this test is unable to distinguish ammonia from other exhaled acidic or basic gases.

Likewise, U.S. patent application Ser. No. 10/617,008 teaches a method for detecting in the gastrointestinal tract of a subject the presence of a bacterium which catalyzes the degradation of urea to ammonia and carbon dioxide, wherein the method involves delivering a source of urea to the subject; inducing the subject to exhale a breath sample so that the breath contacts a sensor (e.g., a dye such as bromophenol blue, bromothymol blue, phenol red, methyl orange, methyl yellow, 2,4-dinitrophenol, 2,6-dinitrophenol, and cresol red which has the capacity to become deprotonated and undergo a color change in the presence of ammonia); and optically detecting a color change in the sensor after the subject exhales into the container. In this test, too, the measurement of the pH response of a single dye is unable to distinguish ammonia from other exhaled acidic or basic gases.

Needed is a non-invasive, cost-efficient, ammonia-selective method and apparatus for detecting the presence of H. pylori infection of the gastrointestinal tract in a subject, which is extremely rapid, sensitive and which can provide real-time results, for example in the context of a single visit to a physician's office. The present invention meets this long-felt need.

### SUMMARY OF THE INVENTION

The present invention is an apparatus for detecting the presence of exhaled ammonia. The apparatus is a breath capture device having a sensor plate with at least one selected Lewis acid dye deposited thereon which produces a detectable spectral, transmission or reflectance response in the presence of ammonia.

The present invention is also a method for detecting the presence of a Helicobacter in a subject. The method involves the steps of obtaining a first exhaled breath in a breath capture device having a sensor plate with at least one selected Lewis acid dye deposited thereon which produces a detectable spectral, transmission or reflectance response in the presence of ammonia; detecting a first spectral, transmission or reflectance response of the selected Lewis acid dye; administering to the subject a specified amount of urea; obtaining a second exhaled breath in said device; detecting a second spectral, transmission or reflectance response of the selected Lewis acid dye; and comparing the first and second spectral, transmission or reflectance response, wherein a difference in the first and second spectral, transmission or reflectance response is indicative of the presence of Helicobacter in the subject.

### DETAILED DESCRIPTION OF THE INVENTION

The present invention relates the use of amine-sensitive dyes, in particular selected Lewis acid dyes (e.g., metalloporphyrin), for detecting the presence of ammonia in exhaled breath. An apparatus and method for using the same are provided which can be advantageously used to detect the presence of a Helicobacter infection or for diagnosing liver or renal function.

In general, an apparatus of the present invention is a breath capture device having a sensor plate, for example as part of a cartridge, with at least one selected amine-sensitive Lewis acid dye deposited thereon. The breath capture device can be any suitable structure that facilitates the passage of the exhaled breath over the sensor plate. For example, the breath capture device can be a mask or conical-shaped structure which the subject exhales a breath into. Alternatively, the breath capture device can be a tube, e.g., similar to an alcohol breathalyzer, or container which can capture volatile components in breath and remove water vapor from the breath to facilitate detection of ammonia. See, for example, U.S. Pat. Nos. 4,749,553; 5,458,853; and 6,726,637, which disclose breath capture devices suitable for use in accordance with the apparatus of the instant invention. When the subject exhales a breath into the breath capture device, the breath device conducts the breath sample into a sensor array cartridge that is mounted on a suitable imaging platform. A downstream pump with a flow meter and regulator can optionally be used to draw the exhaled breath through the sensor array cartridge at specific flow rates. The apparatus can be used at ambient temperature or within several degrees of physiologic temperature for optimal ammonia detection and can be made of a medical grade plastic capable of being initially sterilized and can optionally be disposable.

In general, the sensor plate has deposited thereon at least one selected amine-sensitive Lewis acid dye capable of changing color in the presence of ammonia. In a particular embodiment, the sensor plate has an array of two-dimensionally spatially-resolved dyes, wherein at least one of the dyes is a selected Lewis acid dye. It is contemplated that an array can encompass two, three, four, five or more dyes, wherein all or a portion of the dyes are Lewis acid dyes. An array can alternatively encompass 10, 15, 20, 25, 30, 35, 40, or 50 dyes. While, 1 part per million of ammonia is a physiologically relevant level of ammonia for detecting the presence of a Helicobacter infection or for diagnosing liver function, a sensor plate of the instant invention can, in general, detect as little as 10 parts per billion of ammonia.

A selected Lewis acid dye is defined as a dye which has been identified for its ability to interact with an amine by acceptor-donor sharing of a pair of electrons from the amine resulting in a change in color and/or intensity of color (caused by, e.g., Lewis acid-base interaction, ligand binding, pi-pi complexation, or polarity induced shifts in color) indicating the presence of the amine. In particular embodiments of the present invention, the selected Lewis acid dye interacts with ammonia.

Suitable selected Lewis acid dyes for use in accordance with the apparatus and method disclosed herein include metal ion-containing dyes and Bronsted acid dyes, which, in general, are non-metalated dyes. In one embodiment, the apparatus and method of the instant invention utilize a metal ion-containing Lewis acid dye. Metal ion-containing or three-coordinate boron-containing Lewis acid dyes are generally strongly colored dyes that contain within them a Lewis acid site capable of affecting the color of the dye; the Lewis acid is an electron pair acceptor capable of accepting or sharing a pair of electrons from a Lewis base (i.e., an electron pair donor) such as ammonia. In another embodiment, the apparatus and method of the instant invention utilize a Bronsted acid dye. A Bronsted acid is a proton donor which can change color by donating a proton to a Bronsted base (i.e., a proton acceptor) such as ammonia.

Metal ion-containing Lewis acid dyes include, but are not limited to, metal ion-containing porphyrins (i.e., metalloporphyrins), salen complexes, chlorins, bispocket porphyrins, and phthalocyanines. Particularly suitable metal ions complexed with dyes for detecting ammonia include Zn(II) and Co(III) metals. In particular embodiments of the present invention, the selected Lewis acid dye is a metalloporphyrin. For example, diversity within the metalloporphyrins can be obtained by variation of the parent porphyrin, the porphyrin metal center, or the peripheral porphyrin substituents. The parent porphyrin is also referred to as a free base porphyrin, which has two central nitrogen atoms protonated (i.e., hydrogen cations bonded to two of the central pyrrole nitrogen atoms). A particularly suitable parent porphyrin is 5,10,15,20-tetraphenylporphyrinate(−2) (TPP dianion), its metalated complexes, its so-called free base form (H₂TPP) and its acid forms (H₃TPP⁺ and H₄TPP⁺²). As exemplified herein, selected variants of TPP have been found to interact with an amine and change color. See Example 1. Likewise, a selected TPP variant interacts with ammonia and changes color. See Example 2. Accordingly, suitable selected metal ion-containing metalloporphyrin dyes for use in the apparatus and method of the present invention include, but are not limited to,
2,3,7,8,12,13,17,18-octafluoro-5,10,15,20-tetrakis(pentafluorophenyl)porphyrinatocobalt(II) [Co(F₂₈TPP)];
2,3,7,8,12,13,17,18-octabromo-5,10,15,20-tetraphenylporphyrinatozinc (II) [Zn (Br₈TPP)];
5,10,15,20-tetraphenylporphyrinatozinc(II) [ZnTPP]; 5(phenyl)-10,15,20-trikis(2′,6′-bis(dimethyl-t-butylsiloxyl)phenyl)porphyrinatozinc(II) [Zn(Si₆PP)];
5,10,15,20-tetrakis(2′,6′-bis(dimethyl-t-butylsiloxyl)phenyl)porphyrinatozinc(II) [Zn(Si₈PP];
5,10,15,20-Tetraphenyl-porphyrinatocobalt (II) [CoTPP];
5,10,15,20-Tetrakis(2,6-difluorophenyl)porphyrinatozinc(II) [Zn-F2PP]; and

5,10,15,20-Tetrakis(2,4,6-trimethylphenyl)porphyrinatozinc(II) [ZnTMP].

The synthesis of such porphyrins is well-established in the art and is described in U.S. patent application Ser. No. 10/279,788.

Bronsted acid dyes include, but are not limited to, protonated, but non-metalated, porphyrins, chlorins, bispocket porphyrins, phthalocyanines, and related polypyrrolic dyes. Polypyrrolic dyes, when protonated, are in general pH-sensitive dyes (i.e., pH indicator or acid-base indicator dyes that change color upon exposure to acids or bases) or solvatochromic dyes (i.e., dyes that change color depending upon the local polarity of their microenvironment). In one embodiment, a Bronsted acid dye is a non-metalated porphyrin such as 5,10,15,20-tetrakis(2′,6′-bis(dimethyl-t-butylsiloxyl)phenyl)porphrin dication [H₄Si₈PP]⁺²; 5,10,15,20-Tetraphenyl-21H,23H-porphine [H₂TPP]; or 5,10,15,20-Tetraphenylporphine dication [H₄TPP]⁺². As described in Example 2, selected indicator dyes interact with ammonia and change color. Accordingly, in another embodiment of the instant invention, a selected Bronsted dye is an indicator dye including, but are not limited to, Bromocresol Purple, Cresol Red, Congo Red, Thymol Blue, Bromocresol Green, Nile Red, Bromothymol Blue, Methyl Red, Nitrazine Yellow, Phenol Red, Bromophenol Red, Disperse Orange 25, and Bromophenol Blue. As will be appreciated by the skilled artisan, the Bronsted acids disclosed herein may also be considered Bronsted bases under particular pH conditions. Likewise, a non-metalated, non-protonated, free base form of a bispocket porphyrin may also be considered a Bronsted base. However, these dye forms are also expressly considered to be within the scope of the dyes disclosed herein.

The addition of Bronsted acid dyes to arrays containing metal ion-containing Lewis acid dyes as described herein improves the sensitivity of the array for ammonia and increases the ability to discriminate between analytes. Further, the use of one or more selected metal ion-containing dyes in combination with one or more Bronsted acid dyes can advantageously create a signature indicative of the presence of ammonia. Thus, one particular embodiment of the instant invention is a sensor plate containing at least one selected metal ion-containing Lewis acid dye and at least one selected Bronsted dye. Those skilled in the art will recognize that other modifications and variations in the choice of dyes can be made in addition to those described and illustrated herein without departing from the spirit and scope of the present invention. For example, dyes known to detect a particular analyte found in the breath of a normal healthy breath (i.e., not infected with Helicobacter) can be included in an array of metal ion-containing Lewis acid dyes and Bronsted acid dyes as a positive control.

As will be appreciated by one of skill in the art, an array of dyes for detecting ammonia can be combined on the same sensor plate with an array of dyes which detect other volatile analytes (e.g., alcohol or sulfur) thereby expanding the diagnostic capabilities of the sensor plate for multiple diseases or disorders.

Dyes can be affixed to the surface of the sensor plate by direct deposition, including, but not limited to, airbrushing, ink-jet printing, screen printing, stamping, micropipette spotting, or nanoliter dispensing. The metalloporphyrins, in particular, have excellent chemical stability on a solid support and most have well-studied solution ligation chemistry. Thus, the sensor plate can be made from any suitable material or materials, including but not limited to, chromatography plates, paper, filter papers, porous membranes, or properly machined polymers, glasses, or metals.

It is contemplated that the apparatus of the instant invention can further be combined with a means for monitoring the spectral response, transmission response or reflectance response of the dye(s) on the sensor plate at one or more wavelengths in a spatially resolved fashion. This can be accomplished with an imaging spectrophotometer, a flatbed scanner (e.g., an EPSON® Model 2480 or 4870), slide scanner, a video or CCD or CMOS camera, or a light source combined with a CCD or CMOS detector. When used in combination with arrays of amine-sensitive Lewis acid dyes and image analysis software, calorimetric difference maps can be generated by subtracting the red, green and blue (RGB) values of dye images generated before and after exposure of the dye to a test sample. The calorimetric differences maps represent hue and intensity profiles for the array in response to analytes (e.g., ammonia) in the sample. This eliminates the need for extensive and expensive signal transduction hardware associated with previous techniques (e.g., piezoelectric or semiconductor sensors). When used in accordance with the method of the present invention, a unique color change signature can be created which provides both qualitative recognition and quantitative analysis of ammonia in an exhaled breath.

The apparatus of the present invention can further be combined with standard chemometric statistical analyses (e.g., principal component analysis and hierarchal cluster analysis), an artificial neural network (ANN) for data analysis, or other pattern recognition algorithms of color changes to dyes in response to ammonia. For example, ANN is an information processing system that functions similar to the way the brain and nervous system process information (Tuang, et al. (1999) FEMS Microbiol. Lett. 177: 249-256). The ANN is trained for the analysis and then tested to validate the method. In the training process, the ANN is configured for pattern recognition, data classification, and forecasting. Commercial software programs are available for this type of data analysis. To illustrate, a standardized set of data from an array of dyes exposed to ammonia serves as the input vector. The desired output vector is the classification of ammonia (i.e., Helicobacter), “0” for no ammonia (i.e., no Helicobacter) and “1” for ammonia (i.e., Helicobacter). Training is accomplished by using the standardized data set and associating the input or signature with the desired output or classification. The program compares the data and computes network output with the desired output until an acceptable level of recognition is achieved.

The apparatus disclosed herein can be used for detecting ammonia in exhaled breath for the purposes of diagnosing a Helicobacter infection or disorders associated with the liver or kidney. A number of bacterial strains possess urease activity and colonize within the gastrointestinal tract of a variety of subjects. For example, when the subject is a human, many species of Helicobacter, including H. pylori and H. heilmanii colonize within the gastrointestinal tract (Hirschl (1994) Wien Klin. Worchenschr. 106(17): 538-42) and can cause gastric ulcers. Moreover, when the subject is a primate, H. nemestinae has been show to colonize within the gastrointestinal tract (Solnick and Schauer (2001) Clinical Microbiology Reviews 14(1): 59-97). Further, when the subjects are felines or canines, many distinct species of Helicobacter including H. bizzozeronii, H. salomonis and H. felis colonize within the gastrointestinal tract (Solnick and Schauer (2001) supra).

Bacteria having urease activity that colonize in the gastrointestinal tract of a subject, as described above, have also been linked to disorders associated with the liver. In particular, H. pylori and H. heilmanii have been associated with hepatitis (McCathey, et al. (1999) Helicobacter 4(4): 249-59).

Accordingly, the present invention is also a method for using the apparatus disclosed herein in the detection of a Helicobacter infection. The method involves the steps of obtaining a first exhaled breath in a breath capture device comprising a sensor plate having at least one selected Lewis acid dye deposited thereon which produces a detectable spectral, transmission or reflectance response in the presence of ammonia; detecting a first spectral, transmission or reflectance response of the selected Lewis acid dye (i.e., baseline); administering to the subject a specified amount of urea; obtaining a second exhaled breath in said device; detecting a second spectral, transmission or reflectance response of the selected Lewis acid dye; and comparing the first and second spectral, transmission or reflectance responses, wherein a difference in the first and second spectral, transmission or reflectance responses is indicative of the presence of a Helicobacter in the subject.

In general, exhaled breath samples are obtained from the subject's lungs through the nose, mouth, trachea, or other external orifice of the subject. Typically, and most conveniently, the exhaled breath can be obtained by having the subject exhale (or blow) into a breath capture device, e.g., of the apparatus disclosed herein. For example, the subject can exhale breath into a mask or tube which facilitates contact between the contents of the breath and a sensor plate having at least one selected Lewis acid dye deposited thereon for analysis. Desirably, the subject exhales a breath directly onto the sensor plate. Alternatively, other samples such as saliva and gastric reflux are also contemplated.

To obtain a first exhaled breath sample or baseline, the subject typically exhales for about 1 to about 60 minutes, or more generally about 5 to about 30 minutes into a breath capture device. The exhaled breath sample (i.e., vapor or liquid) is then brought into contact with the ammonia sensor plate and the presence and/or amount of ammonia is subsequently detected by observing, either visually or by optical inspection, a change in the color and/or intensity of color of the Lewis acid dye (i.e., metal ion-containing dye and/or Bronsted acid dye) deposited on the sensor plate. Obtaining and testing of a first exhaled breath sample prior to the administration of urea is particularly advantageous to increase the accuracy of the method. However, it is contemplated that the method of the present invention can be carried out without taking a baseline reading, wherein the results of the breath test can be compared to a set of standardized baseline array data or control array data (e.g., a suitable range of responses collected from subjects known to have a Helicobacter infection).

Once a first spectral response, transmission response or reflectance response of the Lewis acid dye is obtained (i.e., baseline), the subject is administered a specified amount of urea. In general, any source of urea, in solid or liquid form can be administered to the subject. Such sources include any composition that can be converted to ammonia in vivo or otherwise serve as a substrate for urease of the bacteria being detected. By way of example, the urea source can constitute urea, per se, or it can be a derivative of urea.

The amount of the urea administered to the subject is preferably sufficient to produce a detectable concentration of ammonia in an exhaled breath sample obtained from a subject, without undue adverse side effects to the subject, such as toxicity, irritation or allergic responses. For a particular subject, the amount can vary and generally depends upon a variety of factors such as the form of the urea source, the weight of the subject, and species of the subject. In general, however, the amount administered will be from about 1 milligram to about 20 milligrams of urea per kilogram body weight of the subject.

The urea source can be administered to the gastrointestinal tract of the subject by any generally known method. In one embodiment, administration is by oral ingestion of urea, in single or multiple doses. The particular dosage form used to administer the urea can be, for example, in solid tablets or capsules, or in liquid solutions or emulsions. Moreover, urea can be administered essentially in pure form, as detailed herein, or as part of a composition. Compositions useful in administration of urea can also contain pharmaceutically-acceptable components such as, for example, diluents, emulsifiers, binders, lubricants, colorants, flavors and sweeteners. Suitable components included in the composition, however, preferably do not interfere with hydrolysis of the urea, or generate appreciable quantities of ammonia in the gastrointestinal tract. An optional component is one which delays gastric emptying, thereby increasing the length of time that the administered urea is present in the stomach.

After administration of the urea source to the subject, a period of time sufficient for the bacteria to catalyze urea to ammonia and carbon dioxide is allowed to elapse before obtaining the exhaled breath sample. In one embodiment, about 0.1 to about 120 minutes elapse after administration of urea prior to obtaining the second exhaled breath sample. In another embodiment, about 5 to about 60 minutes elapse after administration of urea prior to obtaining the second exhaled breath sample. Subsequently, a second exhaled breath is obtained. To obtain the second exhaled breath, the subject typically exhales for about 1 to about 120 minutes, or more generally about 30 to about 60 minutes into a breath capture device. The second exhaled breath sample is brought into contact with the ammonia sensor plate and the presence and/or amount of ammonia is subsequently detected as described for the first exhaled breath sample. The sensor plate used to detect ammonia in the second exhaled breath can be the same sensor used for detecting ammonia in the first exhaled breath or can be a different sensor plate with the same or different array of dyes as the sensor plate used to detect ammonia in the first exhaled breath.

The first and second spectral, transmission or reflectance responses of the selected Lewis acid dye are then compared. If the second spectral, transmission or reflectance response is different from the first spectral, transmission or reflectance response, as a result of the administration and metabolism of urea, a positive diagnosis of a Helicobacter infection in the subject being tested can be made. By way of illustration, prior to administration of urea, only a nominal amount of ammonia will be present in the exhaled breath sample, e.g., on the order of less than about 1 part per million. After the administration of urea, however, the amount of ammonia present in the exhaled breath sample will increase by a magnitude of about 2-fold, 3-fold, 10-fold to about 1000-fold. Accordingly, by comparing the amount of ammonia present in both pre- and post-urea breath samples, the method of the present invention provides an extremely accurate means to determine the presence of a bacterium possessing urease activity in the gastrointestinal tract of a subject.

As will be appreciated by the skilled artisan, the apparatus and general steps of the method of the instant invention can be readily modified for use in detecting ammonia, or other exhaled amines or analytes in exhaled breath for other medical diagnostic purposes such as assessment of liver or renal function or detection of airway malignancies (e.g., lung cancer), sinus infections (e.g., bacterial or fungal sinusitis), respiratory infections (e.g., pneumonia), and the like. In such assays, the subject may or may not be administered urea or other substrates (e.g., N-alkylamine or other Cytochrome P450 substrate) prior to detection of exhaled ammonia or other analyte and may or may not have a baseline reading taken prior to the administration of the substrate. Wherein a baseline reading is not taken, the results of the breath test can be compared to a set of standardized baseline array data or control array data which are indicative of the particular disease being diagnosed.

The invention is described in greater detail by the following non-limiting examples.

### EXAMPLE 1

### Detection of an Amine in the Presence of Water Vapor

Using an array of TPP variants, the detection of n-hexylamine in the presence of water vapor was analyzed. It was found that a color fingerprint generated from exposure of the array to n-hexylamine (0.86% in N₂) was identical to that for n-hexylamine spiked heavily with water vapor (1.2% H₂O, 0.48% hexylamine in N₂). See Table 1. The ability to easily detect species in the presence of a large water background represents a substantial advantage over mass-sensitive sensing techniques or methodologies that employ polar polymers as part of the sensor plate.

TABLE 1
Hexylamine, neat (0.86% in N₂)

| | | |
|---|---|---|
| Sn⁴⁺ - No Change | Co³⁺ - Green | Cr³⁺ - Green |
| Mn³⁺ - No Change | Fe³⁺ - Red | Co²⁺ - No Change |
| Cu²⁺ - No Change | Ru²⁺ - No Change | Zn²⁺ - Green |
| Ag²⁺ - No Change | 2H⁺ (Free Base) - Dark Blue | |

Hexlamine spiked with water

| | | |
|---|---|---|
| Sn⁴⁺ - No Change | Co³⁺ - Green | Cr³⁺ - Green |
| Mn³⁺ - No Change | Fe³⁺ - Red | Co²⁺ - No Change |
| Cu²⁺ - No Change | Ru²⁺ - Green (small dot) | Zn²⁺ - Green |
| Ag²⁺ - No Change | 2H⁺ (Free Base) - Dark Blue | |

Difference Map

| | | |
|---|---|---|
| Sn⁴⁺ - 0 | Co³⁺ - 0 | Cr³⁺ - 0 |
| Mn³⁺ - 0 | Fe³⁺ - 0 | Co²⁺ - 0 |
| Cu²⁺ - 0 | Ru²⁺ - 0 | Zn²⁺ - 0 |
| Ag²⁺ - 0 | 2H⁺ (Free Base) - 0 | |

“0” indicates that a bluish circle was observed in the digital subtraction of the hexylamine and hexylamine spiked with water scans.

### EXAMPLE 2

### Detection of Humidified Ammonia

A flow-over experimental assembly for ammonia was utilized for testing sensor array responsiveness to humidified ammonia at 1 ppm (part per million). The assembly consisted of an air-tight sensor array cartridge, TEFLON® tubing, gas mixing and metering manifolds in order to deliver controllable and reproducible gas flow to the array. One ppm ammonia was generated by a 100-fold dilution of commercially-certified 100 ppm cylinder of ammonia in nitrogen. The carrier gas used for these studies was water-saturated nitrogen, generated by bubbling a nitrogen stream through water. Control experiments were also performed, wherein sensor arrays were exposed to only water-saturated nitrogen. The array was imaged on a flat-bed scanner before and after exposure. Each analyte (water-saturated nitrogen and 1 ppm ammonia) was tested on triplicate sensor arrays. An array of various metal ion-containing Lewis acid dyes and Bronsted acid dyes was used. Each dye spot was represented by three color channels: Red, Green and Blue (RGB), wherein their chemoresponsive changes can span a theoretical range of 0-255. Accordingly, there are 108 color values associated with the array; R, G and B for each of the 36-spots.

Ammonia was detected as early as 1 minute by the array sensor and dye responses reached their maxima by 10-15 minutes. Color changes of several exemplary dyes exposed to 1 ppm ammonia for 15 minutes are listed in Table 2. RGB responses were reproducible within 5% for 1 ppm ammonia. Responses of dyes to water-saturated nitrogen were negligible.

TABLE 2

| | | R, G, and B Changes for |
|---|---|---|
| Dye | Color Channel | 1 ppm Ammonia |
| Cresol Red | R2 | −1 |
| | G2 | 75 |
| | B2 | −59 |
| BromoPhenol Red | R12 | −69 |
| | G12 | −22 |
| | B12 | 2 |
| Bromocresol Green | R13 | −143 |
| | G13 | −59 |
| | B13 | 44 |
| Methyl Red | R14 | 12 |
| | G14 | 30 |
| | B14 | −30 |
| Phenol Red | R18 | −2 |
| | G18 | 49 |
| | B18 | −39 |
| [H₄TPP]⁺² | R24 | 30 |
| | G24 | −28 |
| | B24 | 27 |
| Disperse Orange 25 | R28 | 0 |
| | G28 | 7 |
| | B28 | −1 |

### EXAMPLE 3

### Detection of H. pylori in a Rat Model

Breath sensing experiments were conducted to determine the ability of ammonia sensing arrays to detect ammonia generated by urea-treated rats infected with H. pylori. A total of 17 animals were tested; 12 had been inoculated with H. pylori and 5 were non-inoculated controls. An array of 36 amine-sensitive Lewis acid dye formulations, including several porphyrins and macrocyclic dyes, was used as the sensor for these studies. Each animal's baseline breath was collected for 20 minutes and ammonia levels detected by the amine-sensitive array. After a pause, to remove the animal from the holder, the animals were administered either a urea or vehicle-only gavage. Subsequently, the animal was replaced in its holder and imaging was resumed for an additional 60 minutes to collect the post-treatment breath on the same amine-sensitive array. Image analysis software was used to extract the numeric RGB vector data for each experiment into a single database.

Experiments having baseline and test results for 5, 10, 15 and 20 minutes were selected for analysis by auto-associative artificial neural network (AA-ANN) modeling and auto-clustering algorithms. The AA-ANN modeling worked with the RGB sensor data to successfully predict the presence of ammonia in the breath of test animals. There were distinct clusters of data for infected animals treated with either 5 mg or 25 mg of a urea solution, while infected animals treated with a non-urea containing gavage (either water only or empty gavage needle) fell into dissimilar separate clusters (Table 3). Likewise, uninfected control animals, gavaged with either vehicle or urea also clustered distinctly with the vehicle-treated infected animals. The results of the AA-ANN auto clustering were verified by microbial culturing of the animals upon necropsy, indicating that the amine-sensitive porphyrin containing arrays can positively detect the presence of a Helicobacter infection. When substrate was present in excess, there was no difference observed regarding clustering. These data also demonstrate that ammonia was produced immediately following administration of urea to H. pylori-infected animals and was detected by the sensor arrays within minutes.

TABLE 3

| | | | | Hidden neuron | | | |
|---|---|---|---|---|---|---|---|
| | | | | outputs | | | |
| Cluster # | Rat # | “infected” | Urea (g/L) | H1 | H2 | H3 | H4 |
| 1 | 1 | 1 | 5 | 1.00 | 0.00 | 0.00 | 1.00 |
| | 2 | 1 | 5 | 1.00 | 0.00 | 0.00 | 1.00 |
| | 6 | 1 | 5 | 1.00 | 0.00 | 0.00 | 1.00 |
| | 15 | 1 | 25 | 1.00 | 0.00 | 0.00 | 1.00 |
| | 16 | 1 | 25 | 0.59 | 0.00 | 0.00 | 1.00 |
| | 17 | 1 | 25 | 1.00 | 0.00 | 0.00 | 1.00 |
| 2 | 3 | 1 | 5 | 1.00 | 1.00 | 0.00 | 0.00 |
| | 4 | 1 | 5 | 1.00 | 1.00 | 0.00 | 0.00 |
| 3 | 7 | 0 | 5 | 1.00 | 0.00 | 1.00 | 1.00 |
| 4 | 3 | 1 | 0 | 0.00 | 1.00 | 1.00 | 0.00 |
| | 11 | 0 | 25 | 0.00 | 1.00 | 1.00 | 0.00 |
| | 6 | 0 | 25 | 0.00 | 1.00 | 1.00 | 0.00 |
| | 3 | 1 | 0 | 0.00 | 1.00 | 1.00 | 0.00 |
| 5 | 4 | 1 | 0 | 0.00 | 1.00 | 0.23 | 0.00 |
| 6 | 9 | 0 | 25 | 0.00 | 0.00 | 0.28 | 1.00 |
| 7 | 10 | 0 | 25 | 0.00 | 0.00 | 0.62 | 1.00 |
| 8 | 12 | 1 | 25 | 1.00 | 1.00 | 0.00 | 1.00 |
| | 13 | 1 | 25 | 1.00 | 1.00 | 0.00 | 1.00 |
| 9 | 14 | 0 | 25 | 0.41 | 0.69 | 0.00 | 1.00 |
| | 1 | 1 | 0 | 0.00 | 1.00 | 0.00 | 1.00 |
| | 7 | 0 | 25 | 0.00 | 1.00 | 0.00 | 1.00 |
| | 8 | 0 | 25 | 0.00 | 1.00 | 0.00 | 1.00 |
| 10 | 2 | 1 | 0 | 0.00 | 0.00 | 1.00 | 0.00 |
| | 10 | 0 | 0 | 0.01 | 0.00 | 1.00 | 0.00 |
| | 2 | 1 | 0 | 0.00 | 0.00 | 1.00 | 0.00 |
| | 11 | 0 | 0 | 0.00 | 0.00 | 1.00 | 0.00 |

Analysis of the auto-clustering revealed that a high output (i.e., 1.00) of the hidden neuron H1 together with a low output of the hidden neuron H3 (i.e., 0.00) indicated the presence of ammonia. Analysis of the cluster's hidden neuron attributes revealed five dye formulations within the 36 spot array that were critical for ammonia identification within rat breath. The intensities of three of the dyes increased when contacted with ammonia, whereas the intensities of the other two decreased when contacted with ammonia.

### EXAMPLE 4

### Detection of H. pylori in Human Subjects

Lewis acid-based testing uses an array of calorimetric dye formulations specifically tuned to detect ammonia with sensitivity to 10 parts per billion (ppb). A baseline breath sample is obtained (e.g., for 15 minutes) and a then the subject consumes a urea preparation. Within 1-5 minutes of urea ingestion, the subject breaths into a tube connected to a breath capture device for 15 minutes. Optionally, Mylanta is consumed to raise the pH of the gastric juice, converting ammonium ions to ammonia. Subjects resume breathing for an additional 15 minutes.

1. An apparatus for detecting the presence of exhaled ammonia comprising a breath capture device comprising a sensor plate having at least one selected Lewis acid dye deposited thereon which produces a detectable spectral, transmission or reflectance response in the presence of ammonia.

2. A method for detecting the presence of a Helicobacter in a subject comprising obtaining a first exhaled breath in a breath capture device comprising a sensor plate having at least one selected Lewis acid dye deposited thereon which produces a detectable spectral, transmission or reflectance response in the presence of ammonia; detecting a first spectral, transmission or reflectance response of the selected Lewis acid dye; administering to the subject a specified amount of urea; obtaining a second exhaled breath in said device; detecting a second spectral, transmission or reflectance response of the selected Lewis acid dye; and comparing the first and second spectral, transmission or reflectance response, wherein a difference in the first and second spectral, transmission or reflectance response is indicative of the presence of Helicobacter in the subject.
